(19) United States
(12) Patent Application Publication
(10) Pub. No.: US 2019/0205970 A1
(43) Pub. Date: Jul. 4, 2019

(54) SYSTEM AND METHOD FOR SECURING PRODUCTS UTILIZING DNA INFORMATION

(71) Applicant: Walmart Apollo, LLC, Bentonville, AR (US)

(72) Inventors: Brian G. McHale, Chadderton Oldham (GB); Todd D. Mattingly, Bentonville, AR (US); Robert L. Cantrell, Herndon, VA (US); John J. O'Brien, Farmington, AR (US)

(21) Appl. No.: 16/191,620

(22) Filed: Nov. 15, 2018

Related U.S. Application Data
(60) Provisional application No. 62/613,475, filed on Jan. 4, 2018.

(51) Int. Cl. G06Q 30/06; G06Q 30/02; G06Q 30/00; G06Q 20/18; A61B 10/00

(52) U.S. Cl. CPC G06Q 30/0639; G06Q 30/0224; A61B 10/0096; G06Q 30/0609; G06Q 20/18; G06Q 30/0185

(57) ABSTRACT

A request is received at each of the transceivers from a human requestor to access or move a product. The request including information concerning a DNA sample of the human requestor that has been voluntarily obtained. At each of a plurality of transceivers, the information concerning the DNA sample is compared to a list of acceptable DNAs at each of the transceivers. When a match exists and when a predetermined number of nodes confirm the match, one of the plurality of electronic nodes sends an electronic control signal to a locking mechanism at the product to unlock the locking mechanism and release the product.

### CROSS-REFERENCE TO RELATED APPLICATION

This application claims the benefit of the following U.S. Provisional Application No. 62/613,475 filed Jan. 4, 2018, which is incorporated herein by reference in its entirety.

### TECHNICAL FIELD

These teachings relate to product security and, more specifically, to allowing the accessing of protected products utilizing voluntarily obtained DNA information.

### BACKGROUND

Various types of products are sold in retail stores or are stored in warehouses. Some of the products are expensive or have other types of value associated with them. Theft of these products is a concern in today's society. The source of the theft can come from a variety of different sources. Unscrupulous customers can attempt to steal the product. Store employees also sometimes attempt to steal products.

Various attempts have been made to prevent product theft. For example, locks have been attached to the products. These locks, however, typically require physical keys to unlock the product. The keys can become lost or are sometimes otherwise unavailable when the product needs to be unlocked and released to a customer. Another problem associated with these previous approaches is that if the keys are stolen, then an unauthorized person can gain immediate access to the products.

### BRIEF DESCRIPTION OF THE DRAWINGS

The above needs are at least partially met through the provision of approaches that provide product security, particularly when studied in conjunction with the drawings, wherein:

FIG. 1 comprises a diagram of a system as configured in accordance with various embodiments of these teachings;

FIG. 2 comprises a flowchart as configured in accordance with various embodiments of these teachings;

FIG. 3 comprises a flowchart as configured in accordance with various embodiments of these teachings;

FIG. 4 comprises an illustration of blocks as configured in accordance with various embodiments of these teachings;

FIG. 5 comprises an illustration of transactions configured in accordance with various embodiments of these teachings;

FIG. 6 comprises a flow diagram in accordance with various embodiments of these teachings;

FIG. 7 comprises a process diagram as configured in accordance with various embodiments of these teachings;

FIG. 8 comprises an illustration of a delivery record configured in accordance with various embodiments of these teachings;

FIG. 9 comprise a system diagram configured in accordance with various embodiments of these teachings.

### DETAILED DESCRIPTION

Generally speaking, a locking mechanism secures a product. A plurality of electronic control nodes, e.g., base stations, each have a ledger of acceptable DNAs that are voluntarily obtained. A person (e.g., an employee of a retail store or a customer at home) requests to unlock the product and voluntarily supplies their DNA for verification to the group of nodes. If the group of nodes agree that the DNA is acceptable (by each of the based stations comparing the DNA from the person to acceptable DNA on a ledger), then one of the nodes (or some other entity) unlocks the package. The product may be alternatively unlocked using a conventional approach (e.g., with a mechanical key).

In many of these embodiments, a system that is configured to prevent the unauthorized access or movement of a retail store product disposed at a location that is accessible to retail store customers is provided. The system includes a locking mechanism and a plurality of control nodes.

The locking mechanism is disposed about a retail store product to prevent unauthorized access to or movement of the product. The plurality of electronic control nodes are disposed across a geographic area at a retail store. Each of the plurality of electronic control nodes includes a transceiver, a database, and a control circuit. Each of the databases stores a ledger of acceptable DNAs associated with individuals that are allowed to access or move the product. The acceptable DNAs have been voluntarily obtained. Each of the transceivers receives a request from a human requestor to access or move the product. The request includes information concerning a DNA sample of the human requestor. The sample is voluntarily obtained. Each of the control circuits compares the information concerning the DNA sample to the acceptable DNAs. When a match exists and when a predetermined number of nodes confirm the match, one of the plurality of electronic nodes sends an electronic control signal to the locking mechanism to unlock locking mechanism and release the product. An alternative mechanical or electronic lock may also be used to unlock the product manually.

In some examples, the locking mechanism comprises a spider cable with a lock. Other examples of locking mechanisms are possible.

In other aspects, the product is disposed on a shelf in a retail store. In other examples, the product is disposed on the shelf of a warehouse. In still other examples, the product is being delivered to a customer at a customer location such as the customer's home. Other examples are possible.

In aspects, the ledger of acceptable DNAs comprises a blockchain ledger. In yet other examples, the product is disposed in a package and the locking mechanism is disposed so as to prevent unauthorized access to the package. In still other examples, the plurality of electronic control nodes are disposed at base stations towers.

In yet other aspects, the system further includes a DNA sample obtaining device that is disposed at the product. The sampling device is configured to obtain a DNA sample from the human requestor. Obtaining the sample in aspects is completely voluntary. In examples, a user can press their thumb or finger against the device and a DNA sample is obtained.

In others of these embodiments, an approach for preventing the unauthorized access or movement of a retail store product disposed at a location that is accessible to retail store customers is provided. A retail store product is locked to prevent unauthorized access to or movement of the product. A plurality of electronic control nodes are disposed across a geographic area at a retail store. Each of the plurality of electronic control nodes includes a transceiver, a database, and a control circuit. Each of the databases stores a ledger of acceptable DNAs (or DNA information) associated with individuals that are allowed to access or move the product. The DNAs or DNA information has been obtained from individuals completely voluntarily.

A request is received at each of the transceivers from a human requestor to access or move the product. The request includes information concerning a DNA sample of the human requestor. At each of the transceivers, the information concerning the DNA sample is compared to the acceptable DNAs. When a predetermined number of nodes confirm the match, one of the plurality of electronic nodes (or some other entity) sends an electronic control signal to the locking mechanism to unlock locking mechanism and release the product.

In other aspects, a customer or a store employee voluntarily provides their DNA as a sample to a store. This DNA information is added to a ledger of the blockchain as an acceptable DNA (allowing the human owner of the DNA access to a package or other privileges). More specifically, the double helix strand information of the DNA is recorded as information in the blockchain.

In one example, the package that has been sent for delivery is unlocked. For instance, a package may have a lock, which is unlocked to open the package and allow access to merchandise within the package.

If the package is sent to a customer, the customer's DNA is obtained in a voluntary approach. The DNA is sent to one or more nodes, each of which compare it to a ledger of acceptable DNAs. If there is a consensus match amongst the stations, the package is unlocked allowing access by the customer.

These approaches also prevent people from leaving stores with unpaid merchandise since the merchandise is locked (e.g., using a spider cable). However, in a store, employees still need to open the package (e.g., at the checkout). Once an employee or customer's DNA is verified by these approaches, then the package can be opened, the customer can pay for the merchandise, and exit the store.

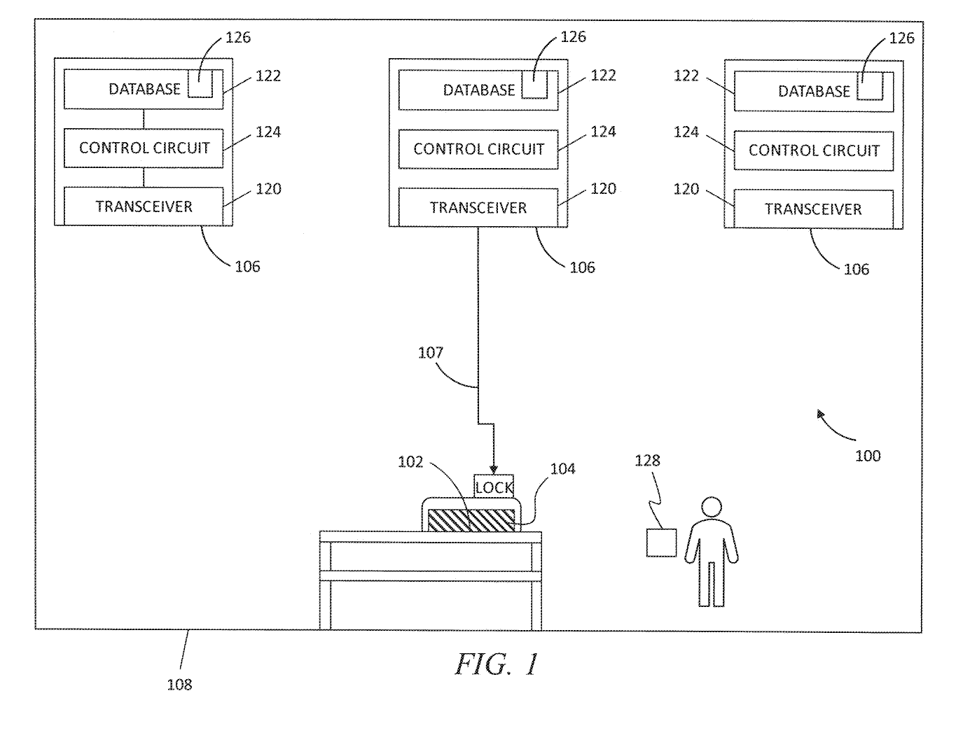

Referring now to FIG. 1, a system 100 that is configured to prevent the unauthorized access or movement of a retail store product disposed at a location that is accessible to retail store customers is described. The system 100 includes a locking mechanism 102 (protecting products 104), and a plurality of electronic control nodes 106.

The locking mechanism 102 is disposed about a retail store product to prevent unauthorized access to or movement of the product. In other aspects, the product is disposed on a shelf in a retail store. In other examples, the product is disposed on the shelf of a warehouse. In still other examples, the product is being delivered to a customer at a customer location such as the customer's home. In yet other examples, the product is disposed in a package. In these examples, a locking mechanism is disposed so as to prevent unauthorized access to the product or package. The locking mechanism 102 may include a mechanical lock that can be opened by, for example, a mechanical key.

The locking mechanism 102 may be any combination of locks, cables, packages, coverings or other devices or structures that prevent unauthorized access to merchandise. In one example, the locking mechanism is a lock that secures a spider cable. In aspects, the locking mechanism 102 includes a receiver that receives electronic control signals that release or unlock the lock. In other examples, the locking mechanism is a lock that opens a package or box. Other examples of locking mechanisms and devices are possible. In still other examples, the locking mechanism includes a door and lock (e.g., where the door secures a storeroom with products stored in the storeroom).

The plurality of electronic control nodes 106 are disposed across a geographic area at a retail store 108. Each of the plurality of electronic control nodes 106 includes a transceiver 120, a database 122, and a control circuit 124. Each of the databases 122 stores a ledger 126 of acceptable DNAs associated with individuals that are allowed to access or move the product. In aspects, the ledger of acceptable DNAs comprises a blockchain ledger. In aspects, a blockchain is a chain of hocks, each with a recorded ledger of validated DNAs. All nodes have a copy of the blockchain which represents the agreed version of the truth.

Each of the transceivers 120 is configured to transmit and receive signals, and each receives a request from a human requestor to access or move the product. The request includes information concerning a DNA sample of the human requestor.

The control circuits 124 are coupled to the transceivers 120 and the databases 122. It will be appreciated that as used herein the term “control circuit” refers broadly to any microcontroller, computer, or processor-based device with processor, memory, and programmable input/output peripherals, which is generally designed to govern the operation of other components and devices. It is further understood to include common accompanying accessory devices, including memory, transceivers for communication with other components and devices, etc. These architectural options are well known and understood in the art and require no further description here. The control circuits 124 may be configured (for example, by using corresponding programming stored in a memory as will be well understood by those skilled in the art) to carry out one or more of the steps, actions, and/or functions described herein.

Each of the control circuits 124 compares the information concerning the DNA sample to the acceptable DNAs (from ledgers 126). When a match exists and when a predetermined number of nodes 106 confirm the match, one of the plurality of electronic nodes 106 sends an electronic control signal 107 to the locking mechanism 102 to unlock locking mechanism 102 and release the product 104.

In yet other examples, the plurality of electronic control nodes 106 are disposed at base station towers. For example, the base station towers may be disposed over a wide geographic area such as a city, region, state, or country. In other examples, the nodes 106 may be disposed in base station-type devices over the area of a store, a warehouse, or a distribution center.

In other aspects, the system 100 further includes a DNA sample obtaining device 128 that is disposed at the product. The sampling device 128 is configured to obtain a DNA sample from the human requestor. Providing the DNA sample is completely voluntary for the human requestor. The sampling device 128 may, in some aspects, obtains the DNA by having a requestor touch the device 128 (to obtain perspiration or skin samples containing the DNA of a user). In other examples, more intrusive techniques (e.g., obtaining a blood sample) may be utilized.

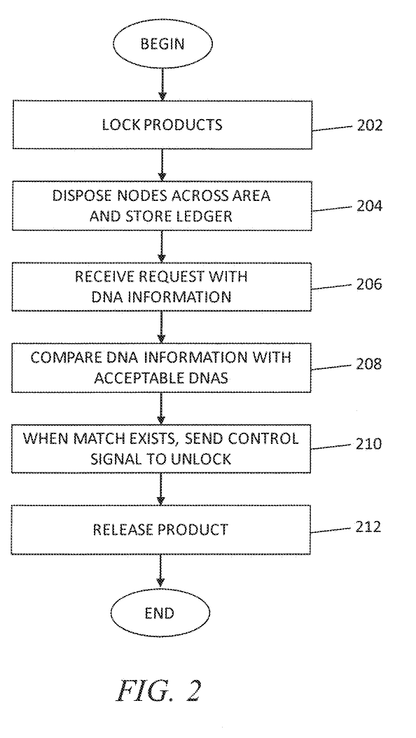

Referring now to FIG. 2, an approach for preventing the unauthorized access or movement of a retail store products disposed at a location that is accessible to retail store customers is described.

At step 202, a retail store product is locked to prevent unauthorized access to or movement of the product. In one example a spider cable with a lock may be used to secure the product. The lock may be associated with or coupled to an electronic wireless communication device that enables the lock to communicate with nodes or base stations.

At step 204, a plurality of electronic control nodes are disposed across a geographic area at a retail store. Each of the plurality of electronic control nodes includes a transceiver, a database, and a control circuit. Each of the databases stores a ledger of acceptable DNAs associated with individuals that are allowed to access or move the product. The nodes or base stations may be disposed over a over a wide geographic area such as a city, region, state, or country. In other examples, the nodes may be disposed in base station-type devices over the area of a store, a warehouse, or a distribution center.

At step 206, a request is received at each of the transceivers from a human requestor to access or move the product. The request includes information concerning a DNA sample of the human requestor. The request may be in any form or format and may include a sample of the DNA of the requestor. The DNA sample may be obtained by any known technique known in the art.

At step 208 and at each of the transceivers, the information concerning the DNA sample is compared to the acceptable DNAs. The acceptable DNAs may be included in any type of blockchain ledger. The acceptable DNAs are preloaded and verified, for example, by a verification service that uses a set of predetermined criteria to determine whether to add a particular DNA to the acceptable list. In aspects, the acceptable DNAs are obtained completely voluntarily from persons.

At step 210, when a predetermined number of nodes confirm the match, one of the plurality of electronic nodes sends an electronic control signal to the locking mechanism to unlock locking mechanism and release the product. Final approval may require the individual approvals of all nodes, a majority of nodes, or a predetermined number of nodes. The locking mechanism can also include a back-up mechanical lock that can be opened manually (e.g., using a key).

At step 212, the product is released when a match has been determined. In examples, an electronic control signal may be sent by one of the nodes to the locking mechanism. When the locking mechanism receives the control signal, the locking mechanism becomes unlocked thereby releasing the product.

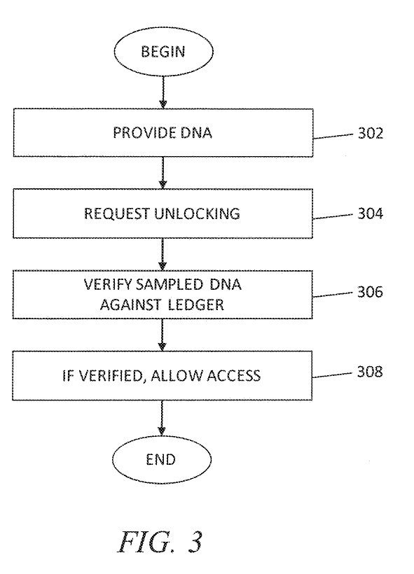

Referring now to FIG. 3, one example of an approach for securing merchandise in a retail store is described. At step 302, a customer or a store employee voluntarily provides their DNA as a sample to a store. This DNA information is added to a ledger of the blockchain as an acceptable DNA (allowing the human owner of the DNA access to a package or other privileges). More specifically, the double helix strand information of the DNA is recorded as information in the blockchain.

At step 304, a customer or store employee requests the unlocking of the merchandise. This request is sent to a node or nodes. The request specifies the person trying to access a package with DNA information of the person (e.g., obtained by a DNA swiping procedure), and determines what the record includes regarding the person.

The nodes (e.g., which may be the base stations) have a file (e.g., a blockchain) of acceptable DNA (e.g., a ledger). Other nodes have the same file. As a request is made to access a package, a message is transmitted to each of the nodes informing them that this person with the DNA wants to open the package. Each node makes a check to determine if the DNA is acceptable. If every node agrees, then the file is updated at each node (and may include the reasons for acceptance).

At step 306, the node or nodes verify their ledger. The nodes may be computers, other retailers (with their own electronic devices that process requests), other locations (with their own electronic devices that process requests), or other customers (with their own electronic devices that process requests). Each node determines whether there is DNA information of the person at their ledger and if so, determines whether the information is acceptable (e.g., determines if there is a match).

At step 308 and if the answer at step 306 is affirmative, then the system allows access to the merchandise. For example, a signal is transmitted to a package to open a package. In another example, a signal is sent to unlock a lock that opens a spider cable. In aspects, store employees could travel behind a register and use a magnetic swipe to open the package or release the product.

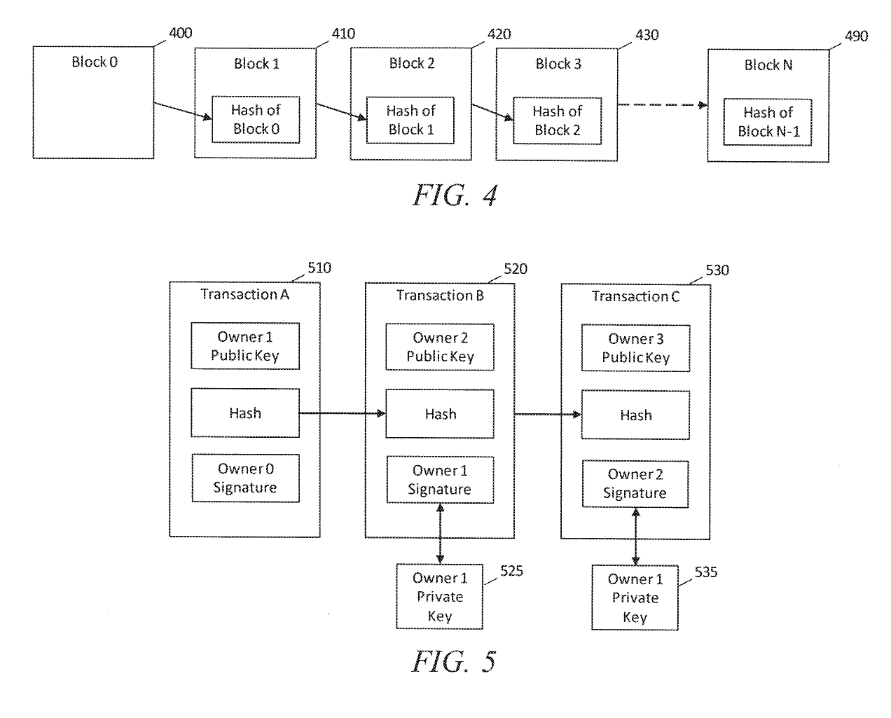

Descriptions of some embodiments of blockchain technology are provided with reference to FIG. 4-9 herein. In some embodiments of the invention described above, blockchain technology may be utilized to record DNA sample information (and other information concerning requestors). One or more of the electronic devices, user devices described herein may comprise a node in a distributed blockchain system storing a copy of the blockchain record. Updates to the blockchain may comprise new DNA information and one or more nodes on the system may be configured to incorporate one or more updates into blocks to add to the distributed database.

Distributed database and shared ledger database generally refer to methods of peer-to-peer record keeping and authentication in which records are kept at multiple nodes in the peer-to-peer network instead of kept at a trusted party. A blockchain may generally refer to a distributed database that maintains a growing list of records in which each block contains a hash of some or all previous records in the chain to secure the record from tampering and unauthorized revision. A hash generally refers to a derivation of original data. In some embodiments, the hash in a block of a blockchain may comprise a cryptographic hash that is difficult to reverse and/or a hash table. Blocks in a blockchain may further be secured by a system involving one or more of a distributed timestamp server, cryptography, public/private key authentication and encryption, proof standard (e.g. proof-of-work, proof-of-stake, proof-of-space), and/or other security, consensus, and incentive features. In some embodiments, a block in a blockchain may comprise one or more of a data hash of the previous block, a timestamp, a cryptographic nonce, a proof standard, and a data descriptor to support the security and/or incentive features of the system.

In some embodiments, a blockchain system comprises a distributed timestamp server comprising a plurality of nodes configured to generate computational proof of record integrity and the chronological order of its use for content, trade, and/or as a currency of exchange through a peer-to-peer network. In some embodiments, when a blockchain is updated, a node in the distributed timestamp server system takes a hash of a block of items to be timestamped and broadcasts the hash to other nodes on the peer-to-peer network. The timestamp in the block serves to prove that the data existed at the time in order to get into the hash. In some embodiments, each block includes the previous timestamp in its hash, forming a chain, with each additional block reinforcing the ones before it. In some embodiments, the network of timestamp server nodes performs the following steps to add a block to a chain: 1) new activities are broadcasted to all nodes, 2) each node collects new activities into a block, 3) each node works on finding a difficult proof-of-work for its block, 4) when a node finds a proof-of-work, it broadcasts the block to all nodes, 5) nodes accept the block only if activities are authorized, and 6) nodes express their acceptance of the block by working on creating the next block in the chain, using the hash of the accepted block as the previous hash. In some embodiments, nodes may be configured to consider the longest chain to be the correct one and work on extending it. A digital currency implemented on a blockchain system is described by Satoshi Nakamoto in “Bitcoin: A Peer-to-Peer Electronic Cash System” (http://bitcoin.org/bitcon. pdf), the entirety of which is incorporated herein by reference.

Now referring to FIG. 4, an illustration of a blockchain according to some embodiments is shown. In some embodiments, a blockchain comprises a hash chain or a hash tree in which each block added in the chain contains a hash of the previous block. In FIG. 4, block 0 400 represents a genesis block of the chain. Block 1 410 contains a hash of block 0 400, block 2 420 contains a hash of block 1 410, block 3 430 contains a hash of block 2 420, and so forth. Continuing down the chain, block N contains a hash of block N−1. In some embodiments, the hash may comprise the header of each block. Once a chain is formed, modifying or tampering with a block in the chain would cause detectable disparities between the blocks. For example, if block 1 is modified after being formed, block 1 would no longer match the hash of block 1 in block 2. If the hash of block 1 in block 2 is also modified in an attempt to cover up the change in block 1, block 2 would not then match with the hash of block 2 in block 3. In some embodiments, a proof standard (e.g. proof-of-work, proof-of-stake, proof-of-space, etc.) may be required by the system when a block is formed to increase the cost of generating or changing a block that could be authenticated by the consensus rules of the distributed system, making the tampering of records stored in a blockchain computationally costly and essentially impractical. In some embodiments, a blockchain may comprise a hash chain stored on multiple nodes as a distributed database and/or a shared ledger, such that modifications to any one copy of the chain would be detectable when the system attempts to achieve consensus prior to adding a new block to the chain. In some embodiments, a block may generally contain any type of data and record. In some embodiments, each block may comprise a plurality of transaction and/or activity records.

In some embodiments, blocks may contain rules and data for authorizing different types of actions and/or parties who can take various actions. In some embodiments, transaction and block forming rules may be part of the software algorithm on each node. When a new block is being formed, any node on the system can use the prior records in the blockchain to verify whether the requested action is authorized. For example, a block may contain a public key of an owner of an asset that allows the owner to show possession and/or transfer the asset using a private key. Nodes may verify that the owner is in possession of the asset and/or is authorized to transfer the asset based on prior transaction records when a block containing the transaction is being formed and/or verified. In some embodiments, rules themselves may be stored in the blockchain such that the rules are also resistant to tampering once created and hashed into a block. In some embodiments, the blockchain system may further include incentive features for nodes that provide resources to form blocks for the chain. For example, in the Bitcoin system, “miners’ are nodes that compete to provide proof-of-work to form a new block, and the first successful miner of a new block earns Bitcoin currency in return.

Now referring to FIG. 5, an illustration of blockchain based transactions according to some embodiments is shown. In some embodiments, the blockchain illustrated in FIG. 5 comprises a hash chain protected by private/public key encryption. Transaction A 510 represents a transaction recorded in a block of a blockchain showing that owner 1 (recipient) obtained an asset from owner 0 (sender). Transaction A 510 contains owner's 1 public key and owner 0's signature for the transaction and a hash of a previous block. When owner 1 transfers the asset to owner 2, a block containing transaction B 520 is formed. The record of transaction B 520 comprises the public key of owner 2 (recipient), a hash of the previous block, and owner 1's signature for the transaction that is signed with the owner 1's private key 525 and verified using owner 1's public key in transaction A 510. When owner 2 transfers the asset to owner 3, a block containing transaction C 530 is formed. The record of transaction C 530 comprises the public key of owner 3 (recipient), a hash of the previous block, and owner 2's signature for the transaction that is signed by owner 2's private key 535 and verified using owner 2's public key from transaction B 220. In some embodiments, when each transaction record is created, the system may check previous transaction records and the current owner's private and public key signature to determine whether the transaction is valid. In some embodiments, transactions are be broadcasted in the peer-to-peer network and each node on the system may verify that the transaction is valid prior to adding the block containing the transaction to their copy of the blockchain. In some embodiments, nodes in the system may look for the longest chain in the system to determine the most up-to-date transaction record to prevent the current owner from double spending the asset. The transactions in FIG. 5 are shown as an example only. In some embodiments, a blockchain record and/or the software algorithm may comprise any type of rules that regulate who and how the chain may be extended. In some embodiments, the rules in a blockchain may comprise clauses of a smart contract that is enforced by the peer-to-peer network.

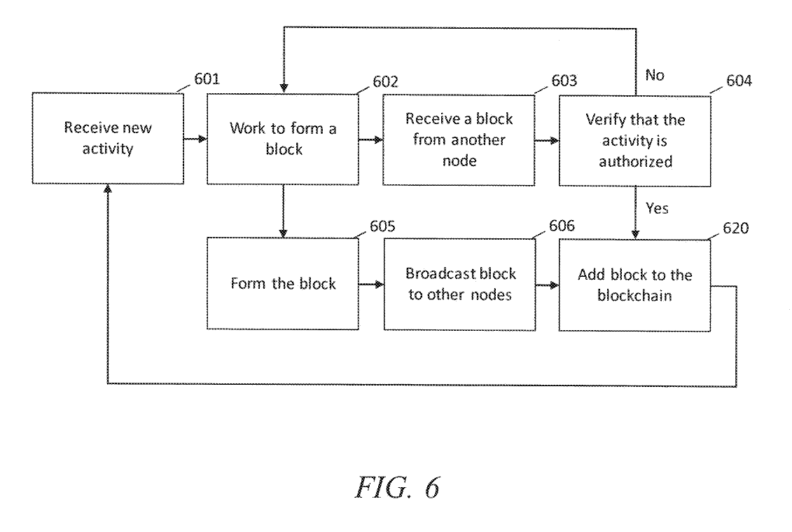

Now referring to FIG. 6, a flow diagram according to some embodiments is shown. In some embodiments, the steps shown in FIG. 6 may be performed by a processor-based device, such as a computer system, a server, a distributed server, a timestamp server, a blockchain node, and the like. In some embodiments, the steps in FIG. 6 may be performed by one or more of the nodes in a system using blockchain for record keeping.

In step 601, a node receives a new activity. The new activity may comprise an update to the record being kept in the form of a blockchain or a request to verify the DNA of a requester against DNA information stored at the blockchain. In some embodiments, for blockchain supported digital or physical asset record keeping, the new activity may comprise a asset transaction. In some embodiments, the new activity may be broadcasted to a plurality of nodes on the network prior to step 601. In step 602, the node works to form a block to update the blockchain. In some embodiments, a block may comprise a plurality of activities or updates and a hash of one or more previous block in the blockchain. In some embodiments, the system may comprise consensus rules for individual transactions and/or blocks and the node may work to form a block that conforms to the consensus rules of the system. In some embodiments, the consensus rules may be specified in the software program running on the node. For example, a node may be required to provide a proof standard (e.g. proof of work, proof of stake, etc.) which requires the node to solve a difficult mathematical problem for form a nonce in order to form a block. In some embodiments, the node may be configured to verify that the activity is authorized prior to working to form the block. In some embodiments, whether the activity is authorized may be determined based on records in the earlier blocks of the blockchain itself.

After step 602, if the node successfully forms a block in step 605 prior to receiving a block from another node, the node broadcasts the block to other nodes over the network in step 606. In some embodiments, in a system with incentive features, the first node to form a block may be permitted to add incentive payment to itself in the newly formed block. In step 620, the node then adds the block to its copy of the blockchain. In the event that the node receives a block formed by another node in step 603 prior to being able to form the block, the node works to verify that the activity recorded in the received block is authorized in step 604. In some embodiments, the node may further check the new block against system consensus rules for blocks and activities to verify whether the block is properly formed. If the new block is not authorized, the node may reject the block update and return to step 602 to continue to work to form the block. If the new block is verified by the node, the node may express its approval by adding the received block to its copy of the blockchain in step 620. After a block is added, the node then returns to step 601 to form the next block using the newly extended blockchain for the hash in the new block.

In some embodiments, in the event one or more blocks having the same block number is received after step 620, the node may verify the later arriving blocks and temporarily store these blocks if they pass verification. When a subsequent block is received from another node, the node may then use the subsequent block to determine which of the plurality of received blocks is the correct/consensus block for the blockchain system on the distributed database and update its copy of the blockchain accordingly. In some embodiments, if a node goes offline for a time period, the node may retrieve the longest chain in the distributed system, verify each new block added since it has been offline, and update its local copy of the blockchain prior to proceeding to step 601.

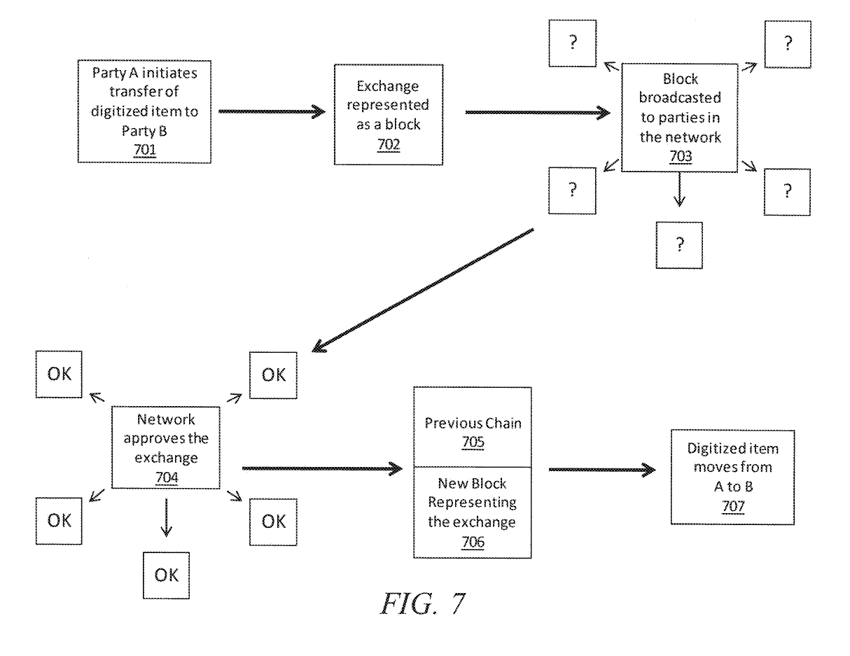

Now referring to FIG. 7, a process diagram a blockchain update according to some implementations in shown. In step 701, party A initiates the transfer of a digitized item to party B. In some embodiments, the digitized item may comprise a digital currency, a digital asset, a document, rights to a physical asset, etc. In some embodiments, Party A may prove that he has possession of the digitized item by signing the transaction with a private key that may be verified with a public key in the previous transaction of the digitized item. In step 702, the exchange initiated in step 701 is represented as a block. In some embodiments, the transaction may be compared with transaction records in the longest chain in the distributed system to verify part A's ownership. In some embodiments, a plurality of nodes in the network may compete to form the block containing the transaction record. In some embodiments, nodes may be required to satisfy proof-of-work by solving a difficult mathematical problem to form the block. In some embodiments, other methods of proof such as proof-of-stake, proof-of-space, etc. may be used in the system. In some embodiments, the node that is first to form the block may earn a reward for the task as incentive. For example, in the Bitcoin system, the first node to provide prove of work to for block the may earn a Bitcoin. In some embodiments, a block may comprise one or more transactions between different parties that are broadcasted to the nodes. In step 703, the block is broadcasted to parties in the network. In step 704, nodes in the network approve the exchange by examining the block that contains the exchange. In some embodiments, the nodes may check the solution provided as proof-of-work to approve the block. In some embodiments, the nodes may check the transaction against the transaction record in the longest blockchain in the system to verify that the transaction is valid (e.g. party A is in possession of the asset he/she s seeks to transfer). In some embodiments, a block may be approved with consensus of the nodes in the network. After a block is approved, the new block 706 representing the exchange is added to the existing chain 705 comprising blocks that chronologically precede the new block 706. The new block 706 may contain the transaction(s) and a hash of one or more blocks in the existing chain 705. In some embodiments, each node may then update their copy of the blockchain with the new block and continue to work on extending the chain with additional transactions. In step 707, when the chain is updated with the new block, the digitized item is moved from party A to party B.

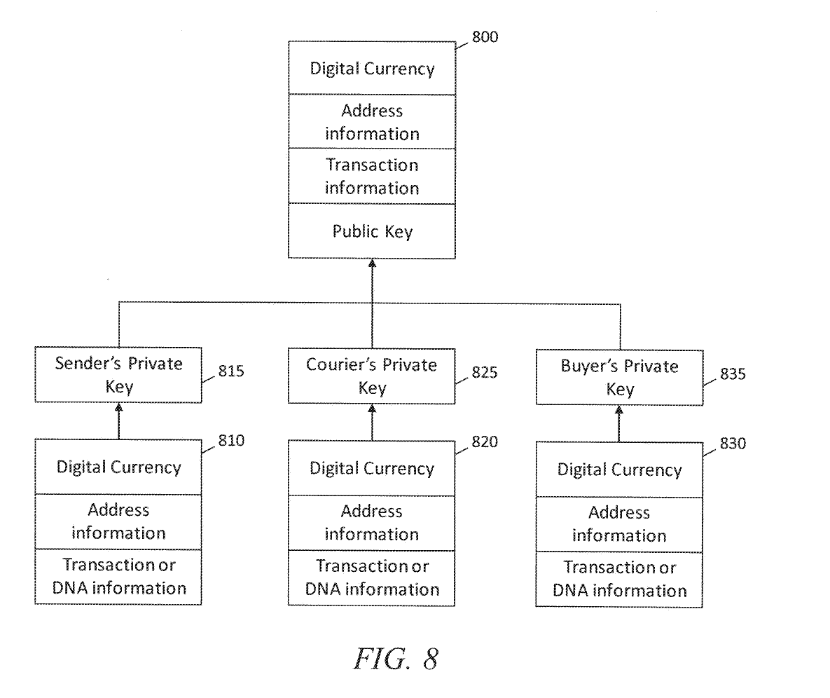

Now referring to FIG. 8, a diagram of a blockchain according to some embodiments in shown. FIG. 8 comprises an example of an implementation of a blockchain system for DNA record keeping. In aspects, the record 800 comprises DNA information, address information, transaction information, and a public key associated with one or more of a requestor, a sender, a courier, and a buyer. In some embodiments, nodes associated the sender, the courier, and the buyer may each store a copy of the record 810, 820, and 830 respectively. In some embodiments, the record 800 comprises a public key that allows the sender, the courier, and/or the buyer to view and/or update the record 800 using their private keys 815, 825, and the 835 respectively.

With the scheme shown in FIG. 8, the record may be updated by one or more of the requester, the sender, courier, and the buyer to form a record of the transaction without a trusted third party while preventing unauthorized modifications to the record. In some embodiments, the blockchain based transactions may further function to include transfers of digital currency with the completion of the transfer of physical asset. With the distributed database and peer-to-peer verification of a blockchain system, the requester, the sender, the courier, and the buyer can each have confidence in the authenticity and accuracy of the DNAs stored in the form of a blockchain.

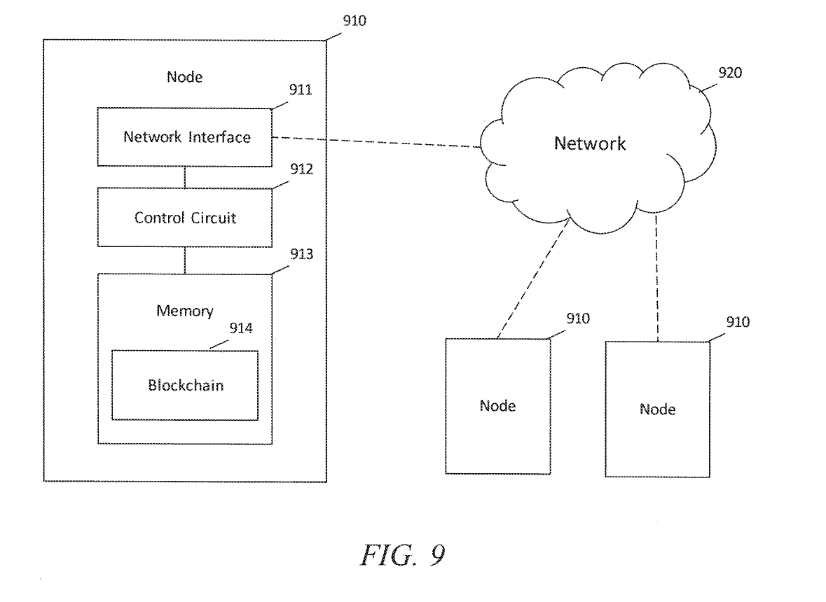

Now referring to FIG. 9, a system according to some embodiments is shown. A distributed blockchain system comprises a plurality of nodes 910 communicating over a network 920. In some embodiments, the nodes 910 may be comprise a distributed blockchain server and/or a distributed timestamp server. In some embodiments, one or more nodes 910 may comprise or be similar to a “miner” device on the Bitcoin network. Each node 910 in the system comprises a network interface 911, a control circuit 912, and a memory 913.

The control circuit 912 may comprise a processor, a microprocessor, and the like and may be configured to execute computer readable instructions stored on a computer readable storage memory 913. The computer readable storage memory may comprise volatile and/or non-volatile memory and have stored upon it a set of computer readable instructions which, when executed by the control circuit 912, causes the node 910 update the blockchain 914 stored in the memory 913 based on communications with other nodes 910 over the network 920. In some embodiments, the control circuit 912 may further be configured to extend the blockchain 914 by processing updates to form new blocks for the blockchain 914. Generally, each node may store a version of the blockchain 914, and together, may form a distributed database. In some embodiments, each node 910 may be configured to perform one or more steps described with reference to FIGS. 6-7 herein.

The network interface 911 may comprise one or more network devices configured to allow the control circuit to receive and transmit information via the network 920. In some embodiments, the network interface 911 may comprise one or more of a network adapter, a modem, a router, a data port, a transceiver, and the like. The network 920 may comprise a communication network configured to allow one or more nodes 910 to exchange data. In some embodiments, the network 920 may comprise one or more of the Internet, a local area network, a private network, a virtual private network, a home network, a wired network, a wireless network, and the like. In some embodiments, the system does not include a central server and/or a trusted third-party system. Each node in the system may enter and leave the network at any time.

With the system and processes shown in, once a block is formed, the block cannot be changed without redoing the work to satisfy census rules thereby securing the block from tampering. A malicious attacker would need to provide proof standard for each block subsequent to the one he/she seeks to modify, race all other nodes, and overtake the majority of the system to affect change to an earlier record in the blockchain.

In some embodiments, blockchain may be used to support a payment system based on cryptographic proof instead of trust, allowing any two willing parties to transact directly with each other without the need for a trusted third party. Bitcoin is an example of a blockchain backed currency. A blockchain system uses a peer-to-peer distributed timestamp server to generate computational proof of the chronological order of transactions. Generally, a blockchain system is secure as long as honest nodes collectively control more processing power than any cooperating group of attacker nodes. With a blockchain, the transaction records are computationally impractical to reverse. As such, sellers are protected from fraud and buyers are protected by the routine escrow mechanism.

In some embodiments, a blockchain may use to secure digital documents such as DAN information, digital cash, intellectual property, private financial data, chain of title to one or more rights, real property, digital wallet, digital representation of rights including, for example, a license to intellectual property, digital representation of a contractual relationship, medical records, security clearance rights, background check information, passwords, access control information for physical and/or virtual space, and combinations of one of more of the foregoing that allows online interactions directly between two parties without going through an intermediary. With a blockchain, a trusted third party is not required to prevent fraud. In some embodiments, a blockchain may include peer-to-peer network timestamped records of actions such as accessing documents, changing documents, copying documents, saving documents, moving documents, or other activities through which the digital content is used for its content, as an item for trade, or as an item for remuneration by hashing them into an ongoing chain of hash-based proof-of-work to form a record that cannot be changed in accord with that timestamp without redoing the proof-of-work.

In some embodiments, in the peer-to-peer network, the longest chain proves the sequence of events witnessed, proves that it came from the largest pool of processing power, and that the integrity of the document has been maintained. In some embodiments, the network for supporting blockchain based record keeping requires minimal structure. In some embodiments, messages for updating the record are broadcast on a best-effort basis. Nodes can leave and rejoin the network at will and may be configured to accept the longest proof-of-work chain as proof of what happened while they were away.

In some embodiments, a blockchain based system allows content use, content exchange, and the use of content for remuneration based on cryptographic proof instead of trust, allowing any two willing parties to employ the content without the need to trust each other and without the need for a trusted third party. In some embodiments, a blockchain may be used to ensure that a digital document was not altered after a given timestamp, that alterations made can be followed to a traceable point of origin, that only people with authorized keys can access the document, that the document itself is the original and cannot be duplicated, that where duplication is allowed and the integrity of the copy is maintained along with the original, that the document creator was authorized to create the document, and/or that the document holder was authorized to transfer, alter, or otherwise act on the document.

As used herein, in some embodiments, the term blockchain may refer to one or more of a hash chain, a hash tree, a distributed database, and a distributed ledger. In some embodiments, blockchain may further refer to systems that uses one or more of cryptography, private/public key encryption, proof standard, distributed timestamp server, and inventive schemes to regulate how new blocks may be added to the chain. In some embodiments, blockchain may refer to the technology that underlies the Bitcoin system, a “sidechain” that uses the Bitcoin system for authentication and/or verification, or an alternative blockchain (“altchain”) that is based on bitcoin concept and/or code but are generally independent of the Bitcoin system.

Descriptions of embodiments of blockchain technology are provided herein as illustrations and examples only. The concepts of the blockchain system may be variously modified and adapted for different applications.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept.

What is claimed is:

1. A system that is configured to prevent the unauthorized access or movement of a retail store product disposed at a location that is accessible to retail store customers, the system comprising:
a locking mechanism that is disposed about a retail store product to prevent unauthorized access to or movement of the product;
a plurality of electronic control nodes that are disposed across a geographic area at a retail store, wherein each of the plurality of electronic control nodes includes a transceiver, a database, and a control circuit;
wherein each of the databases stores a ledger of acceptable DNAs associated with individuals that are allowed to access or move the product, the acceptable DNAs being obtained voluntarily from the individuals;
wherein each of the transceivers receives a request from a human requestor to access or move the product, the request including information concerning a DNA sample of the human requestor that has been obtained voluntarily from the human requestor;
wherein each of the control circuits compares the information concerning the DNA sample to the acceptable DNAs, and
wherein when a match exists and when a predetermined number of nodes confirm the match, one of the plurality of electronic nodes sends an electronic control signal to the locking mechanism to unlock locking mechanism and release the product;
wherein the locking mechanism also includes a back-up mechanical lock that can manually be actuated to release the product.

2. The system of claim 1, wherein the locking mechanism comprises a spider cable.

3. The system of claim 1, wherein the product is disposed on a shelf in a retail store.

4. The system of claim 1, wherein the ledger of acceptable DNAs comprises a blockchain ledger.

5. The system of claim 1, wherein the product is disposed in a package and the locking mechanism is disposed so as to prevent unauthorized access to the package.

6. The system of claim 1, wherein the plurality of electronic control nodes are disposed at base stations towers.

7. The system of claim 1, further comprising a DNA sample obtaining device that is disposed at the product, and is configured to obtain a DNA sample from the human requestor.

8. A method for preventing the unauthorized access or movement of a retail store product disposed at a location that is accessible to retail store customers, the method comprising:
locking a retail store product to prevent unauthorized access to or movement of the product;
disposing a plurality of electronic control nodes across a geographic area at a retail store, wherein each of the plurality of electronic control nodes includes a transceiver, a database, and a control circuit;
wherein each of the databases stores a ledger of acceptable DNAs associated with individuals that are allowed to access or move the product, the acceptable DNAs being obtained voluntarily from the individuals;
receiving a request at each of the transceivers from a human requestor to access or move the product, the request including information concerning a DNA sample of the human requestor that has been obtained voluntarily from the human requestor;
at each of the transceivers, comparing the information concerning the DNA sample to the acceptable DNAs, and
wherein when a match exists and when a predetermined number of nodes confirm the match, one of the plurality of electronic nodes sends an electronic control signal to the locking mechanism to unlock locking mechanism and release the product;
wherein the locking mechanism also includes a back-up mechanical lock that can manually be actuated to release the product.

9. The method of claim 8, wherein the locking uses a spider cable.

10. The method of claim 8, wherein the product is disposed on a shelf in a retail store.

11. The method of claim 8, wherein the ledger of acceptable DNAs comprises a blockchain ledger.

12. The method of claim 8, wherein the product is disposed in a package and the locking prevents unauthorized access to the package.

13. The method of claim 8, wherein the plurality of electronic control nodes are disposed at base stations towers.

14. The method of claim 8, further comprising obtaining the DNA sample from a DNA sample obtaining device.
